(19) United States
(12) Patent Application Publication
(10) Pub. No.: US 2016/0000900 A1
(43) Pub. Date: Jan. 7, 2016

(54) OLIGOMERIC INFLUENZA IMMUNOGENIC COMPOSITIONS

(71) Applicant: FUTUREWEI TECHNOLOGIES, INC., Plano, TX (US)

(72) Inventors: Yusen Zhou, Beijing (CN); Guangyu Zhao, Beijing (CN); Shibo Jiang, New York, NY (US); Lanying Du, New York, NY (US)

(21) Appl. No.: 14/766,093

(22) PCT Filed: Feb. 14, 2014
(86) PCT No.: PCT/US2014/016475

§ 371 Date: Aug. 5, 2015

Related U.S. Application Data
(60) Provisional application No. 61/765,519, filed on Feb. 15, 2013.

(51) Int. Cl. A61K 39/145; C12N 9/10; C12N 9/82; C07K 14/005; C12N 7/00

(52) U.S. Cl. CPC A61K 39/145; C07K 14/005; C12N 7/00; C12N 9/82; C12N 9/1048; C07K 2319/00; C12N 2760/16122; C07K 2319/30; C12N 2760/16134; C12Y 305/01001

(57) ABSTRACT

Embodiments are provided for network resource allocation considering user experience, satisfaction, and operator interest. An embodiment method by a network component for allocating network resources includes evaluating, for a user, a QoE for each flow of a plurality of flows in network traffic in according with a QoE model, and further evaluating, for an operator, a revenue associated with the flows in accordance with a revenue model. A plurality of priorities that correspond to the flows are calculated in accordance with the QoE for the user and the revenue for the operator. The method further includes identifying a flow of the flows with a highest value of the priorities, and allocating a network resource for the flow. In an embodiment, the QoE model is a satisfaction model that provides a measure of user satisfaction for each flow in accordance with a subscription or behavior class of the user.

This application claims the benefit of U.S. Non-Provisional application Ser. No. 14/181,160 filed on Feb. 14, 2014 by Alireza Sharifian et al. entitled “System and Method for Network Resource Allocation Considering User Experience, Satisfaction and Operator Interest,” U.S. Provisional Application No. 61/764,895 filed on Feb. 14, 2013 by Alireza Sharifian et al. entitled “System and Method for Joint Packet/Service Scheduling,” and U.S. Provisional Application No. 61/764,903 filed on Feb. 14, 2013 by Alireza Sharifian et al. entitled “System and Method for User Satisfaction Modeling for Radio Resource Management in Wireless Communications,” which are hereby incorporated herein by reference as if reproduced in their entirety.

### TECHNICAL FIELD

The present invention relates to the field of network optimization, and, in particular embodiments, to a system and method for network resource allocation considering user experience, satisfaction and operator interest.

### BACKGROUND

The next generation of networks is expected to be integrated into broadcasting systems, where content producers/services (such as Hulu, Netflix, Skype, interactive gaming, interactive video, interactive remote robotic control, remote-teaching, telemedicine, etc.) and their customers need stringent assured quality of service (QoS). Currently, QoS is implemented primarily via over-provisioning. Operators are interested in maximizing revenue, providing differentiated services to different users based on their willingness to pay, and ensuring fairness when providing services among the users, e.g., who subscribe to same packages or services. Fairness is a factor to ensure user satisfaction, which is important to service operators in the competitive environment. There is a need for an efficient system and scheme for managing services and network resources according to joint objectives or criteria, such QoS requirement, user fairness, and operator revenue.

### SUMMARY OF THE INVENTION

In accordance with an embodiment, a method by a network component for modeling user satisfaction for network services includes determining quality of service (QoS) requirements for a network service, and measuring QoS elements in network traffic of the network service. The method further includes mapping the measured QoS elements and the QoS requirements to a satisfaction indicator according to a satisfaction model, and determining user satisfaction according to the satisfaction indicator.

In accordance with another embodiment, a network component for modeling user satisfaction for network service includes at least one processor and a non-transitory computer readable storage medium storing programming for execution by the at least one processor. The programming includes instructions to determine QoS requirements for a network service, and measure QoS elements in network traffic of the network service. The programming also includes instructions to map the measured QoS elements and the QoS requirements to a satisfaction measure according to a satisfaction model, and determine user satisfaction according to the satisfaction measure.

In accordance with another embodiment, a method by a network component for allocating network resources includes evaluating, for a user, a QoE for each flow of a plurality of flows in network traffic in according with a QoE model, evaluating, for an operator, a revenue associated with the flows in accordance with a revenue model, and calculating priorities corresponding to the flows in accordance with the QoE for the user and the revenue for the operator. The method further includes identifying a flow of the flows with a highest value of the priorities, and allocating a network resource for the flow.

In accordance with yet another embodiment, a network component for allocating network resources includes at least one processor and a non-transitory computer readable storage medium storing programming for execution by the at least one processor. The programming includes instructions to evaluate, for a user, a QoE for each flow of a plurality of flows in network traffic in according with a QoE model, evaluate, for an operator, a revenue associated with the flows in accordance with a revenue model, and calculate priorities corresponding to the flows in accordance with the QoE for the user and the revenue for the operator. The programming also includes instructions to identify a flow of the flows with a highest value of the priorities, and allocate a network resource for the flow.

The foregoing has outlined rather broadly the features of an embodiment of the present invention in order that the detailed description of the invention that follows may be better understood. Additional features and advantages of embodiments of the invention will be described hereinafter, which form the subject of the claims of the invention. It should be appreciated by those skilled in the art that the conception and specific embodiments disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawing, in which:

FIG. 1 illustrates an embodiment of a framework for multi-objective joint packet/resource scheduling in a network;

FIG. 2 illustrates an embodiment scheme for multi-objective joint packet/resource scheduling;

FIG. 3 illustrates another embodiment of a framework for multi-objective joint packet/resource scheduling;

FIG. 4 illustrates an embodiment of an algorithm including user satisfaction modeling as part of multi-objective joint packet/resource scheduling;

FIG. 5 illustrates an embodiment of a user satisfaction model as part of a multi-objective joint packet/resource scheduling framework;

FIG. 6 illustrates another embodiment of a user satisfaction model as part of a framework multi-objective joint packet/resource scheduling framework;

FIG. 7 illustrates examples of user satisfaction functions.

FIG. 8 illustrates an embodiment of a user satisfaction model as part of a wire line scenario;

FIG. 9 illustrates a user satisfaction model as part of a relay forwarding scenario for wireless networks;

FIG. 10 illustrates an embodiment method for user satisfaction modeling for network services;

FIG. 11 illustrates an embodiment method for multi-objective joint packet/resource scheduling including user satisfaction modeling; and

FIG. 12 is a diagram of a processing system that can be used to implement various embodiments.

Corresponding numerals and symbols in the different figures generally refer to corresponding parts unless otherwise indicated. The figures are drawn to clearly illustrate the relevant aspects of the embodiments and are not necessarily drawn to scale.

### DETAILED DESCRIPTION OF ILLUSTRATIVE EMBODIMENTS

The making and using of the presently preferred embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

In communications systems, user satisfaction depends on both the quality of service requirements (QoS) of an application and on the user's expectation, subscription level, and/or behavior. Typically, radio resource management (RRM) in wireless networks is implemented to provide QoS requirements for different services and users. Additionally, network operators may monitor or consider user satisfaction for delivered services at higher network layers such as the application layer. This typically comprises focusing on one or individual aspects of user satisfaction, such as throughput and or delay, by assessing each aspect individually. Such approaches for managing network resources to provide services with fairness to different users can be challenging, for example when delivering heterogeneous contents to users (e.g., with different packet delays/arrival times, or bit-rate changes over time) over heterogeneous wireless links. Further, the users' applications and service requirements can change over time. The challenges also include the use of different monetary models for different user subscription levels, priority, and/or willingness to pay.

Embodiments are provided herein for network resource allocation considering user experience, satisfaction, and operator interest. The presented schemes also ensure fairness to users when managing resources to provide network services. The embodiments include using a user satisfaction model, or interchangeably a dissatisfaction model, to calculate a global satisfaction (or dissatisfaction) value for multiple users, services, classes of users or services, or traffic flows. The global value is calculated according to a model that takes into consideration various aspects of user satisfaction (or dissatisfaction), such as throughput and delay. The global value can then be fed as an input into a resource allocation or optimization engine of the network. Multiple models corresponding to different classes of users and behaviors can also be used. The embodiments also include a system framework for allocating or scheduling resources based on the satisfaction (or dissatisfaction) value. The framework also takes into consideration monetary models, QoS requirements, and possibly other fairness criteria employed by the network operators for scheduling the resources. The satisfaction, operator charging, and QoS models are combined and used to optimize resource scheduling, e.g., as part of RRM at the physical (PHY) or media access control (MAC) layer, which can improve the efficiency of resource management in heterogeneous traffic and user class scenarios, for example. The optimization results can also be fed back into the system to adjust the different models, further improving the implementation over time. The system framework and satisfaction modeling can be used to efficiently manage users' satisfaction in various timescales and their satisfaction fairness, in addition to conventional service fairness to ensure customer loyalty. The systems also include revenue-awareness to maximize the operator's profit. The embodiments can be implemented by any suitable network technology, including wireless networks, wireline (or fix wired) networks, and other queue/server based networks.

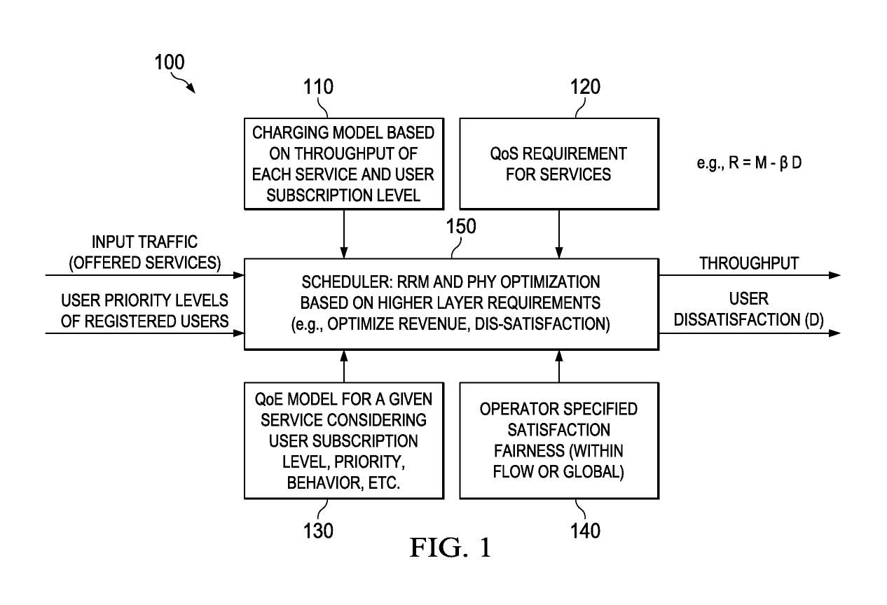

FIG. 1 shows an embodiment of a system 100 for multi-objective joint packet/resource scheduling in a network. The network may be a wireless or cellular network or any other suitable network technology. The system 100 is a multi-objective aware optimization framework for RRM according to operator requirements and user satisfaction relative to their subscription level and service requirements. The multi-objective aware optimization framework joins the operator (seller) and customer interests for RRM. The multiple objectives of this optimization include QoS requirements, user satisfaction fairness, intra-user subscription or behavior class fairness, and monetary aspects for the operator. The system 100 can efficiently incorporate revenue, user satisfaction, generalized fairness (including satisfaction fairness, and intra-class fairness) and QoS requirements into resource and service scheduling.

The system 100 comprises a charging model 110 based on throughput of each service and user subscription level, QoS requirement information 120 for services, a user quality of experience (QoE) model 130 for each service (or each class/type of service) considering user subscription, priority, behavior, or other user relevant classification, and optionally operator specified satisfaction fairness (or dissatisfaction) fairness criteria 140 (e.g., within flow or global). The models or objectives above are fed as input into a scheduler function 150, e.g., at the any queue/server network in general, or at a base station in the case of a wireless network, and used as parameters or values for RRM and PHY/MAC layer optimization, which may be further based on higher layer requirements, e.g., optimize combined revenue and satisfaction. The scheduler function 150 can also use as input additional information such as the amount of traffic for offered services and user priority levels. The output of the optimization by the scheduler function 150 includes revenue and/or user satisfaction indicators, R and D respectively. The output can be in the form of a parameter or a single value for each objective, revenue and user satisfaction (or dissatisfaction) reflecting a level of fairness. The indicators or parameters can also be used to calculate a final utility value, e.g., R−D where is a scaling factor. The utility value is a measure of suitability or overall performance and can be compared to target values.

The scheduling algorithm of the scheduler function 150 is revenue aware to maximize operator revenue, and fairness aware, e.g., to guarantee service ubiquity in all locations within a cell. The charging model 110 can provide input to the scheduler function 150 by making the profit value of each flow transparent to the RRM. The concept of fairness herein is linked to user satisfaction (or dissatisfaction). The QoE model 130 evaluates the current total QoE of flows, balances them, and provides the scheduler function 150 with a final global measure of all considered flows of users. The fairness to users can be addressed by ensuring both satisfaction fairness (in comparison to traditional fairness, where one QoS is balanced to get fairness), and intra-class fairness which provides an additional level of fairness within different QoS classes in a heterogeneous manner based on operators and costumers interest. The algorithm is also made channel aware, to improve transmission opportunities (e.g., service ubiquity and link equalization), and QoS aware to guarantee flow requirement and user satisfaction. Improving user satisfaction ensures a degree of fairness and can prevent users from unsubscribing or leaving the system. The scheduling algorithm can also be utilization/congestion aware to improve the load situation.

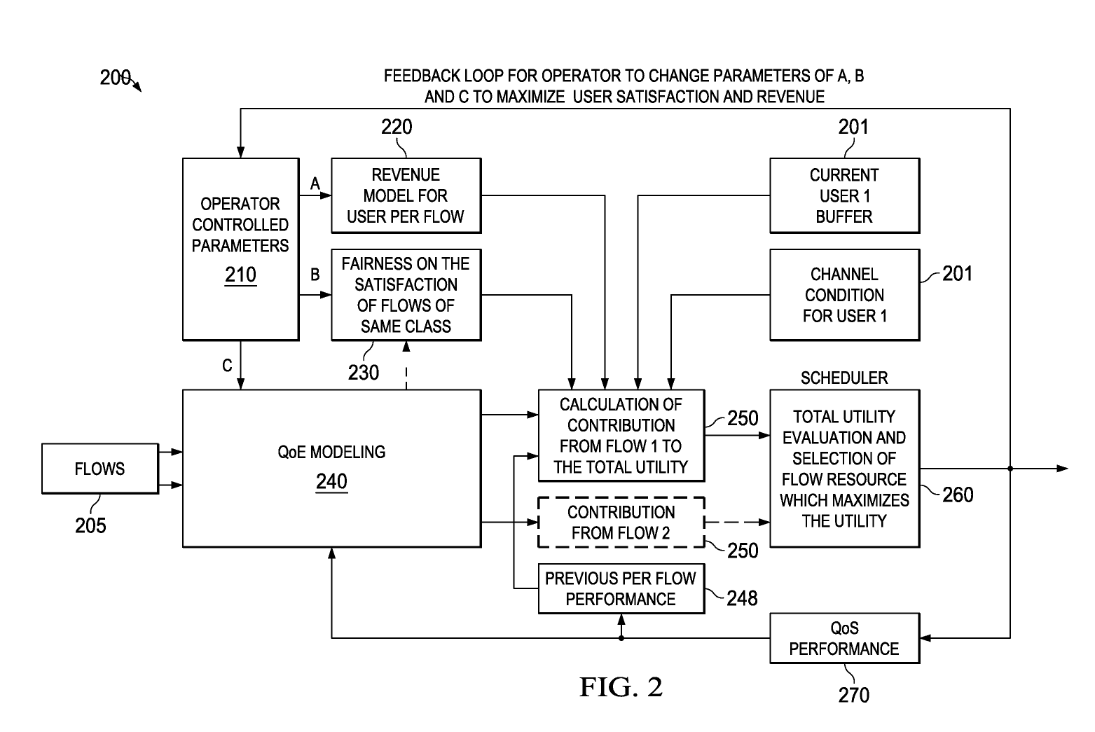

FIG. 2 shows an embodiment scheme 200 for the multi-objective joint packet/resource scheduling of the system 100. The scheme 200 includes calculating, using a per-flow utility calculation function 250 of the network, the contribution for each flow to the network utility. The inputs to per-flow utility calculation function 250 include an output from the revenue model 220 for user per flow, an output from a fairness function 230 on the satisfaction of flows of same class, and an output parameter per flow of a QoE model 240. The QoE model 240 evaluates the user QoE for each one of a plurality of flows 205. For instance, the QoE model 240 can be a satisfaction model configured to calculate a satisfaction value for each one of the flows 205, and provide the calculated value for each flow as an input to the function 250. The output from each of the models/functions above can be in the form of a single parameter or value. The inputs of the per-flow function 250 can also include channel condition information 201 per user and current user buffer information 201 per user. The function 250 calculates the utility per flow using its inputs and sends the utility value per flow as an input to a total utility evaluation function 260. The total utility function 260 evaluates the utility and selects the flow and resource block (RB) which maximize the utility for scheduling for a given resource. Maximizing the utility can be accomplished using mathematical evaluation, which can provide a priority metric for the flows. The priority metric is evaluated to select the best flow. The output of the function 260, e.g., the selected flow and RB, is then allocated to the given resource. The functions 260 and 250 can be part of a scheduler function at a RRM unit.

The function 260 also provides feedback information to the operator to adjust control parameters 210 (A, B, C) for the models/functions 220, 230, and 240. The parameters are used to adjust the respective models to improve revenue, charging scheme, fairness, and user satisfaction. In addition to the feedback information, the parameters can be selected by the operator according to the users' expectations, subscription levels, and/or behaviors. The operator can also use multiple parameters to adjust any one of the models, e.g., to adjust functions in the models. The function 260 can also provide feedback to a QoS performance tracking function 270. The function 270 provides QoS parameters, e.g., per flow, to the QoE model 240. The QoS parameters are used by the QoE model 240 to provide the output per flow to the utility 250. The QoS performance tracking function 270 can also provide historic or previous per flow performance information to the per flow utility calculation function 250. The operator can decide the parameters A, B and C based on an optimization of an internal utility according to inputs such as revenue from each subscription, current resource usage and user experience, the impact of user satisfaction in the long-run, a penalty for not making the expected QoE for the user, and competition and demand for the services. As an example for adjusting the charging scheme, the operator continually monitors the output (consumed) bit-rate and the variation of its server capacity, while serving different QoS classes. The relative price/bit for different QoS classes is then adjusted (feedback loop “A”) based on a relative fraction of these capacities. In this way, the operator can decrease the price/bit sent to the network to reduce the priority for a QoS class at the lower layer, e.g., upon detecting that the relative capacity usage is higher than the relative revenue obtained from that class.

The system 200 allows the operator control of user satisfaction or dissatisfaction level per flow. To schedule resources and services, the system 200 further takes into consideration the fairness of dissatisfaction per same flow class, revenue per flow based on user priority and subscription, channel condition and current buffer size, and past performance and QoS requirements, in addition to intra-class fairness. The framework can also unify real-time (RT) and none real-time packet (RTN) switched connections through different satisfaction functions on different QoS elements, including mean and instantaneous measurements. This unification also enables the system to define handle wider soft differentiated services that are a combination of RT and NRT services.

In an embodiment, the QoE model 240 is a dissatisfaction (or satisfaction) model configured mathematically to calculate fairness in terms of dissatisfaction (or satisfaction), e.g., rather than a raw or single QoS value, in addition to intra-class fairness. The fairness in terms of dissatisfaction can be calculated within the same class of flows. The dissatisfaction functions or equations of the model can also be defined taking into account the importance and priority of each user/flow to achieve satisfaction across different flows/users/services. For example, the dissatisfaction (or can be converted to satisfaction) per flow and frame k is calculated using the

$$function {D}_{φ} [ k ] = {D}_{φ}^{QoS} [ k ] {D}_{φ}^{f} ( {D}_{φ}^{QoS} [ k - 1 ] - \frac{1}{\vert {C}_{φ} \vert} {\sum }_{{φ}^{′} ∈ {C}_{φ}} {D}_{{φ}^{′}}^{QoS} [ k - 1 ] ) where {D}_{φ}^{QoS} [ k ]$$

is the QoS dissatisfaction (or satisfaction) value, D_{φ}^{f} is the dissatisfaction (or can be converted to satisfaction) fairness value, and

$$\frac{1}{\vert {C}_{φ} \vert}$$

is the inverse of the number of flows of a particular same class.

Using the calculated dissatisfaction or satisfaction, the scheduler algorithm or function 260 maximizes a revenue per flow function R_{φ} as follows

$$max {\sum }_{φ} {R}_{φ} ( {r}_{φ} [ k ] , {D}_{φ} ( {QoS}_{φ}^{req} , {QoS}_{φ}^{measur} [ k ] ) ) ,$$

where r_{φ}[k] is the bit-rate per flow and frame k, QoS_{φ}^{req} is the required QoS per flow, and QoS_{φ}^{measur}[k] is the measured QoS per flow and frame k. Examples of the revenue per flow functions include R_{φ}(.)=M_{φ}(r_{φ}[k], QoS_{φ}^{req})−βD_{φ}(QoS_{φ}^{req}, QoS_{φ}^{measur}[k]) where M_{φ} is a price policy function for charging, and

$${R}_{φ} ( . ) = {M}_{φ} ( {r}_{φ} [ k ] , {QoS}_{φ}^{req} ) \frac{{D}_{φ}^{nominal}}{{D}_{φ} ( {QoS}_{φ}^{req} , {QoS}_{φ}^{measur} [ k ] )} ,$$

where D_{φ}^{nominal} is a defined tolerated dissatisfaction value for the flow. The example above is for defining R_{φ}, with regards to price policy and dissatisfaction functions. However, the scheme is not limited to this specific definition of R_{φ}.

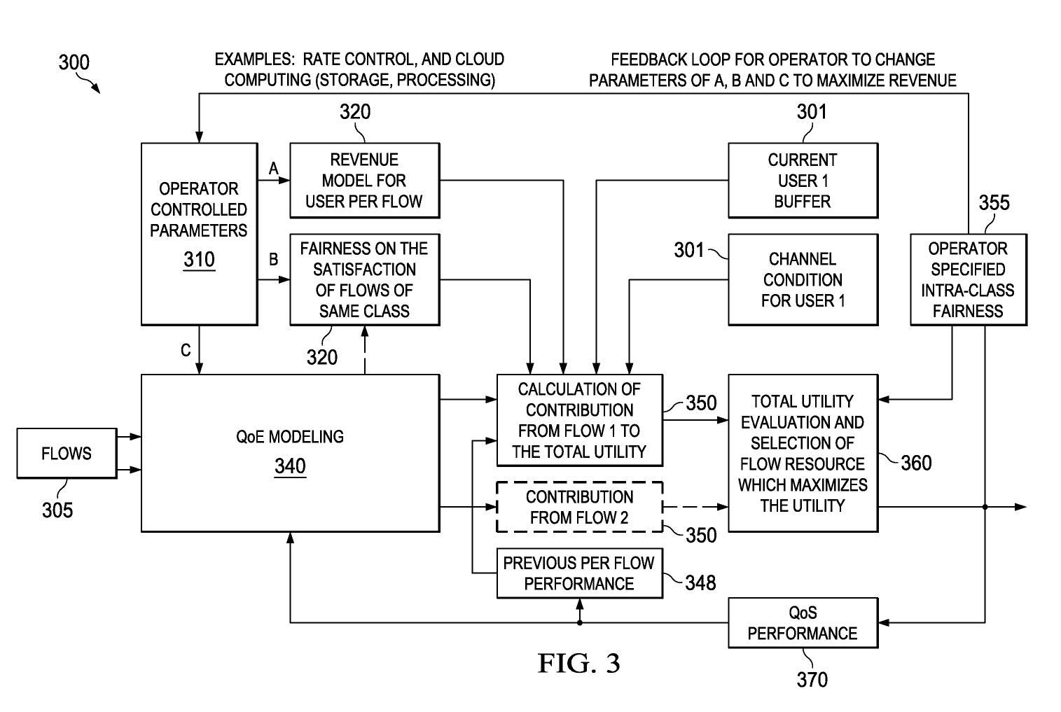

FIG. 3 shows another embodiment scheme 300 for the multi-objective joint packet/resource scheduling of the system 100. Similar to the scheme 200, the scheme 300 calculates, using a per-flow utility calculation function 350 of the network, the contribution for each flow to the network utility. The inputs to per-flow utility calculation function 350 include an output from the revenue model 320 for user per flow, an output from a fairness function 330 on the satisfaction of flows of same class, and a QoE model 340 per each one of a plurality of flows 305. The inputs of the per-flow function 350 can also include channel condition information 301 per user and current user buffer information 301 per user. The function 350 calculates the utility per flow using its inputs and sends the utility value to a total utility evaluation function 360.

The total utility function 360 considers the calculated utilities for all considered flows and selects one or more joint pair of RBs and flows that maximize the overall network utility and satisfy other requirements. The output of the function 360 is then considered to allocate network resources, e.g., data rates and bandwidth. The function 360 also provides feedback information to the operator to adjust control parameters or scaling factors 310 (A, B, C) for the models/functions 320, 330, and 340. The function 360 can also provide feedback to a QoS performance tracking function 370, which provides QoS parameters, e.g., per flow, to modify the QoE model 340. The QoS performance tracking function 370 can also provide historic or previous per flow performance information to the per flow utility calculation function 350. Further, according to the feedback information form the function 360, the operator can use a defined satisfaction fairness, and intra-class fairness model to provide a fairness parameter, e.g., for a higher layer such as the application layer, to the function 360 to evaluate the total utility.

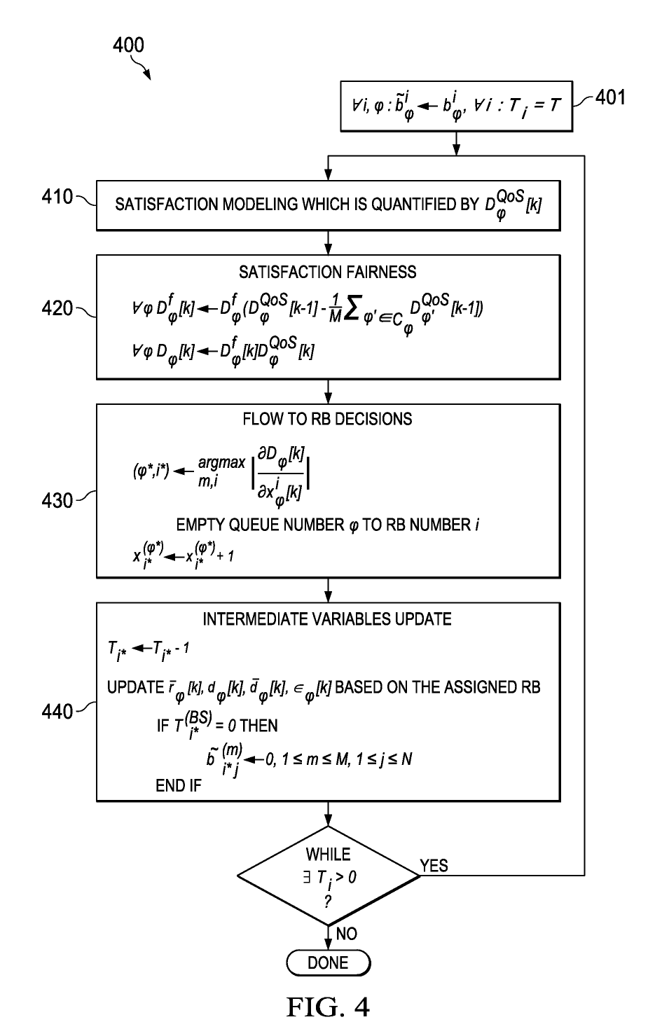

FIG. 4 shows an embodiment of an algorithm 400 for multi-objective joint packet/resource scheduling using user satisfaction modeling. The algorithm 400 can be implemented by the systems 100, for example. At step 401, the bit rates \tilde{b}_{φ}ⁱ for all sub-channels i for each flow are set to measured values b_{φ}ⁱ, and the time slots Tᵢ for each sub-channel i is set to a predetermined value T.

At step 410, the QoS dissatisfaction is quantified according to a dissatisfaction or satisfaction model. For example, a dissatisfaction value is calculated per flow and per frame k. At step 420, for each flow, the fairness dissatisfaction is calculated, e.g., based on the QoS fairness and the function

$$∀ φ {D}_{φ}^{r} [ k ] ← {D}_{φ}^{f} ( {D}_{φ}^{QoS} [ k - 1 ] - \frac{1}{\vert {C}_{φ} \vert} {\sum }_{{φ}^{′} ∈ {C}_{φ}} {D}_{{φ}^{′}}^{QoS} [ k - 1 ] ) . A final$$

dissatisfaction is then calculated as a function of the fairness and QoS dissatisfaction values, e.g., as a product of the two values. Step 430 allocates flows to resource blocks (RBs) to reduce the dissatisfaction (or increase satisfaction), for instance as an argument to maximize a change in dissatisfaction with respect to change in number of used RBs. At step 440, the time slot for each sub-channel is reduced by one, and a plurality of parameters or variables for calculating the dissatisfaction functions are updated. For example, the values include the measured mean bit-rate r̅_{φ}[k] instantaneous delay d_{φ}[k], mean delay d̅_{φ}[k], and/or packet loss ε_{φ}[k] per flow and frame k. If the time slot for a base station (BS) is reduced to zero, the associated bit rates are set to zero. The algorithm is ended if the time slot for each sub-channel is reduced to zero, or returns to step 401 otherwise.

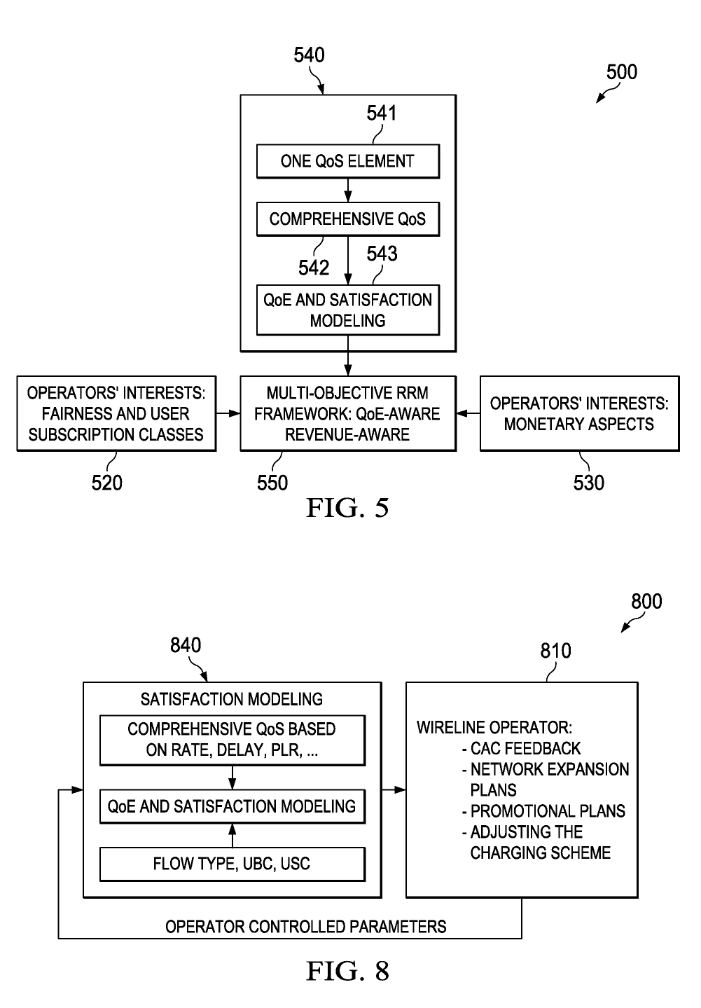

FIG. 5 shows an embodiment of a satisfaction (or dissatisfaction) model 540 as part of a multi-objective joint packet/resource scheduling 500. The model 540 comprises multiple calculation steps for calculating satisfaction or dissatisfaction, leading to a final or global value for the model 540. Each step can use a defined function and the results from a previous step. At a first step 541, a QoS satisfaction is calculated for each user. Each user may have a corresponding configured QoS satisfaction function for calculating its QoS satisfaction value, e.g., according to user expectation, subscription level, and/or behavior. At a second step 542, the calculated QoS satisfaction values for individual users are combined (e.g., as a sum or weighted sum) into a comprehensive satisfaction value. At a third step 543, a combined QoE and satisfaction value is calculated based on the comprehensive QoS satisfaction. This is one possible example of a satisfaction model 540. Other examples may include less or more calculation steps. In another example, at the first step, both QoS satisfaction and QoE satisfaction can be calculated for each user using corresponding functions. In yet another example, an intermediate step between the step 541 and 542 can be used to combine the QoS satisfactions for different subgroups of users (e.g., different behavior classes) using corresponding functions. The final result of the model 540 is then fed into a multi-objective joint packet/resource scheduling framework 550, such as described in the systems above. The framework 550 may also obtain inputs for different fairness and user classes 520 and different monetary aspects 530. Theses inputs may be used to compute corresponding outputs using the results from the model 540. Further, a same or different model 540 may be used each flow or service type.

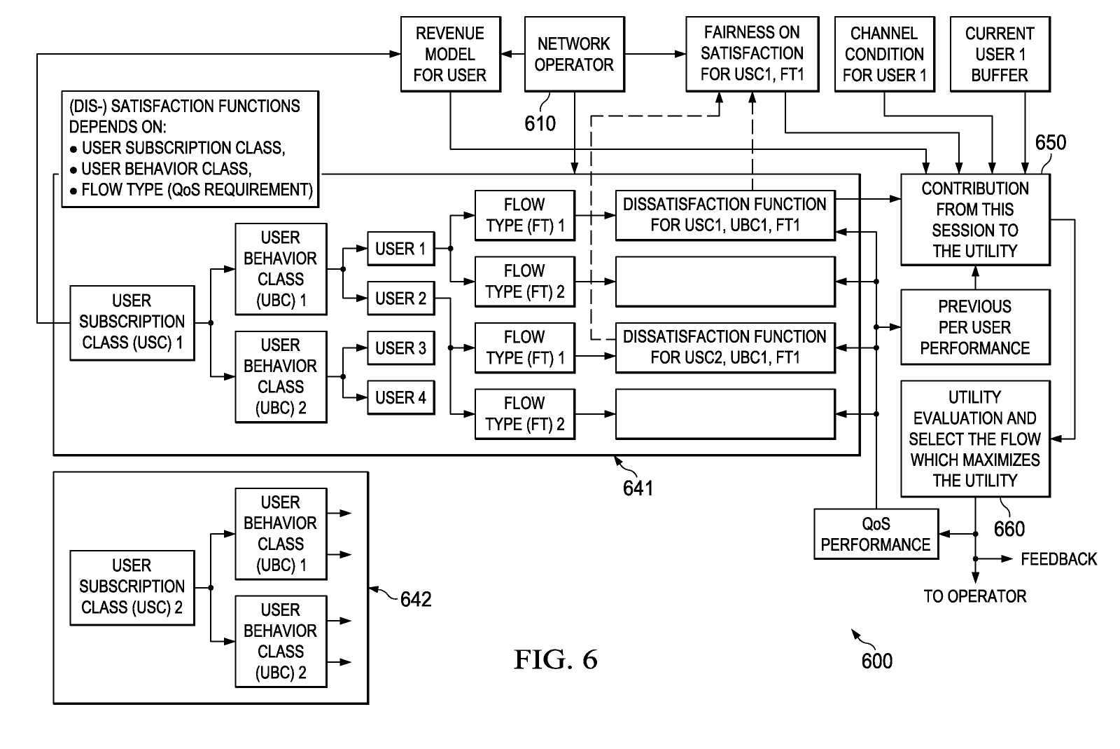

FIG. 6 shows another embodiment of a satisfaction (or dissatisfaction) model 641 as part of a multi-objective joint packet/resource scheduling 600. The model 641 provides multiple satisfaction values for different groupings of users to a per session utility calculation function 650, which calculates a utility value from the session using the values form the model 641. The values are calculated in the model 641 using respective satisfaction or dissatisfaction functions. The inputs of model 641 include user behavior class (UBC) and user service class (USC), in addition to QoS elements. The satisfaction or dissatisfaction functions can be adjusted by the operator 610 using corresponding parameters 610 according to feedback from a utility evaluation and flow selection function 660. The components of the system 600 can be configured similar to the respective components of the systems above.

In the model 641, the users (associated with the session) are separated into groups and further subgroups, e.g., according to user subscription classes, user priority, behavior and flow type, and/or operators dynamic requirements. A (dis)satisfaction value can be calculated according to a tailored function for each subgroup. For example, the operator can classify users based on their behavior of dissatisfaction. This may be obtained through their complaints, surveys, or other user feedback. This classification is then used to modify the dissatisfaction functions. Users can also be further classified according to their flow types and subscription classes. For example, some users may be subscribed with a flat rate service in a gold priority class, while other users may be pay-as-go subscribers in gold priority class. These two sets of users can have different (dis)satisfaction functions.

The operator 610 can dynamically change the function parameters in the model 641 to reflect the user subscription classes, user priority, behavior and flow type, and/or operators dynamic requirements. The operator 610 can also provide different parameters for the dissatisfaction/satisfaction functions as apriori information to the RRM network unit or a network scheduler function, which implements the per session utility calculation function 650. When a flow with a given subscription class is considered, the user priority RRM unit changes the function accordingly. Similarly, a suitable model can be used for each session. For example, a second model 642 is used for a second session.

Defined QoS and QoE parameters can be used to calculate the satisfaction/dissatisfaction values. The parameters allow modifying the calculation functions in the model/system with ease of implementation in lower network layers (e.g., without the need to substitute the calculation functions). In an embodiment, for each given QoS/QoE parameter, a user satisfaction function is defined, resulting in a tailored combined dissatisfaction function. For example, considering delay, when the 5% delay is substantially above the delay bound, the dissatisfaction could be zero. However, when the 5% delay is getting closer to the delay bound, the dissatisfaction increases rapidly. If the delay surpasses the expected or determined delay bound, there is additional penalty in dissatisfaction.

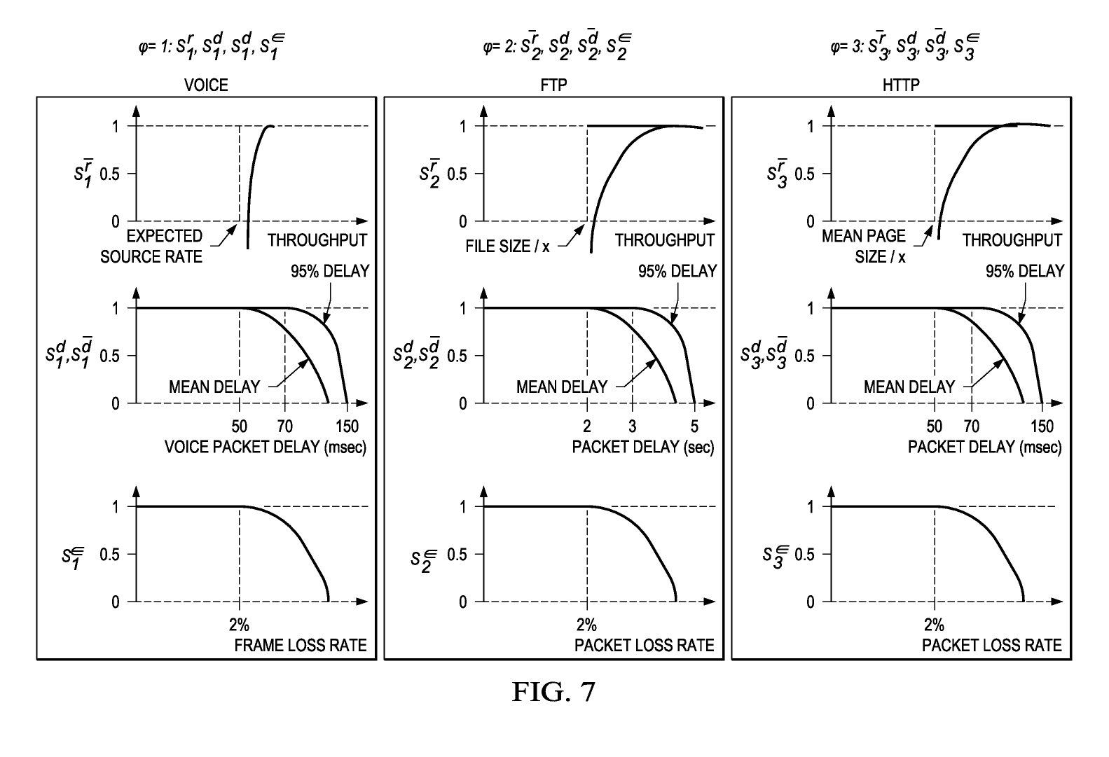

FIG. 7 shows examples of user satisfaction functions, which can be used in the systems above to calculate satisfaction or other related values. The functions are satisfaction functions corresponding to Voice, File Transfer Protocol (FTP), and Hypertext Transfer Protocol (HTTP) services. Four QoS elements form a QoS vector, including rate instantaneous delay, mean delay, and packet loss. The relative effectiveness of each satisfaction function corresponding to a flow can be controlled using parameters corresponding to the four elements as detected or measured. A separate function for each service or a combined function of all services can be calculated. Each calculated value for a flow can be obtained as a combination (e.g., a product or sum) of individual functions of the four elements. The parameters and hence the functions can be modified by the operator dynamically.

As an example, a QoS satisfaction function D_{φ}^{QoS}[k] of frame k per flow □□can be defined as a product of four functions of the four elements above as D_{φ} ^{r̅}( r̅_{φ}[k]−r_{φ}^{min})×D_{φ}^{d}(d_{φ}[k]−d_{φ}^{max})×D_{φ} ^{d̅}( d̅_{φ}[k]− d̅_{φ}^{max})×D_{φ}^{ε}(ε_{φ}[k]−ε_{φ}^{max}). The function D_{φ} ^{r̅}( r̅_{φ}[k]−r_{φ}^{min}) is the bit-rate r̅_{φ}[k] dissatisfaction with respect to its expected minimum r_{φ}^{min}. The function D_{φ}^{d}(d_{φ}[k]−d_{φ}^{max}) is the instantaneous head-of-line (HOL) delay d_{φ}[k] dissatisfaction with respect to its expected minimum d_{φ}^{max}. The function D_{φ} ^{d̅}( d̅_{φ}[k]− d̅_{φ}^{max}) is the mean delay d̅_{φ}[k] dissatisfaction with respect to its expected maximum d̅_{φ}^{max}. The function D_{φ}^{ε}(ε_{φ}[k]−ε_{φ}^{max}) is the packet loss ratio ε_{φ}[k] dissatisfaction with respect to its expected minimum ε_{φ}^{max}. The satisfaction model can further include a user satisfaction function to consider further elements such as jitters or other issues that could affect user satisfaction.

In another example, the model can be extended to handle both RT and RNT traffic in a common pool of resources as described below. For RT flows, the gradients of dissatisfaction functions are obtained as

$$\frac{\partial {D}_{φ} [ k ]}{\partial {x}_{φ}^{( j )} [ k ]} = {( {b}_{φ}^{( j )} )}^{k} {}_{φ}^{{d}^{HOL}} ( {d}_{φ}^{HOL} [ k ] ) ,$$

if φεΦ_{RT}, where κ is the channel-awareness exponent of RT flows, and F_{φ}^{d}^{^{HOL}} is a positive non-decreasing function which represents HOL-delay importance. For NRT flows, the gradients of dissatisfaction functions are evaluated as

$$\frac{\partial {D}_{φ} [ k ]}{\partial {x}_{φ}^{( j )} [ k ]} = min ( ξ , {b}_{φ}^{( j )} [ k ] \frac{{}_{φ}^{\overline{q}} ( {\overline{q}}_{φ} [ k ] )}{{}_{φ}^{\overline{r}} ( {\overline{r}}_{φ} [ k ] )} ) ,$$

if φεΦ_{NRT}, where ξ is a sliding factor between the complete separation of RT and NRT flow and the common pool of resources, q̅_{φ} is the mean queue-length of flow φ until frame k, r̅_{φ} is the mean bit-rate of flow φ until frame k, and F_{φ} ^{q̅}, F_{φ} ^{r̅} are mean queue-length and mean bit-rate importance functions, respectively. The parameters can be used in a closed loop control system to achieve a target level of bit-rate fairness. The parameters and functions relationship can be adjusted, based on operators need, to control various trade-off in different QoS efficiencies and fairness scenarios.

In some scenarios, the QoS of different flows for a service can have different impacts to the overall user satisfaction of that service. This could be integrated by changing the parameters of different flows, for example. Different user subscription classes may also have different dissatisfaction functions for the same service, which allows adjusting resources of different users to match their requirements. This can also include user priority differentiation. Service providers can examine user behaviors and satisfy different users with different level of quality degradation for the same service/flow type by a QoS to QoE mapping. This can be used for example to control video buffering and interruptions. When each flow type has specified QoS requirement, the satisfaction can be mapped as a soft function of the required QoS. The mapping can be further modified for the UBC and/or USC.

The satisfaction modeling can be implemented in other scenarios than the framework described above. FIG. 8 illustrates an embodiment of a satisfaction model 840 as part of a wire line system 800. The satisfaction model 840 allows a wireline (e.g., cable or digital subscriber line (DSL)) operator 810 to adjust resources, such as data rate, delay, packet loss rate (PLR), or other elements. The operator 810 can thus map QoS requirement to user satisfaction according to detected user behavior. The model 840 includes determining comprehensive QoS based on the rate or other elements, and accordingly calculate QoE satisfaction. The model may consider further factors, such as flow type, UBC, and USC. The operator 810 uses the satisfaction value from the model 840 to evaluate satisfaction and performance, and uses feedback to adjust the model's parameter. The feedback information can include, cost of access control, network expansion plans, promotional plans, or adjustments to the charging scheme, for example.

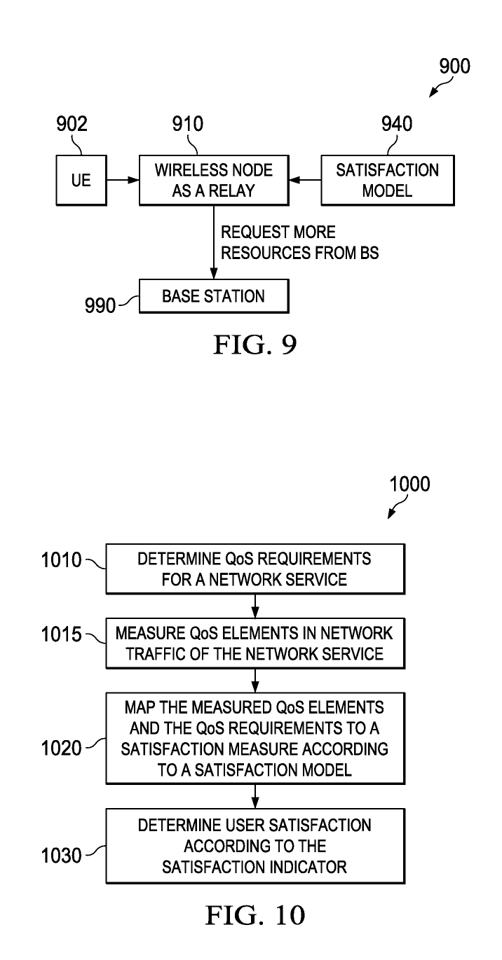

FIG. 9 illustrates an embodiment of a satisfaction model 940 as part of a relay forwarding system 900 for wireless networks. The satisfaction model 940 allows a wireless node or relay 910 to adjust resources for a user or request more resources from the network or a base station 990. The adjustments can include power control, inter-cell interference coordination (ICIC), cell switch-off decisions, user priority determination, or configuring users as relays for other users, for example. Alternatively, the satisfaction model 940 can be implemented at the base station 990 or network.

In general, the satisfaction modeling can be extended to any suitable queue/server model scenarios, such as in wireless communications, wireline communications, cloud computing, power grid optimization, or other network services. As such, the definition of QoS elements, flow, and RB depends on the scenario of implementation.

FIG. 10 illustrates an embodiment method 1000 for user satisfaction modeling for network services. At step 1010, one or more QoS requirements are determined for a network service. At step 1015, one or more QoS elements are measured in network traffic of the network service. The measured QoS elements correspond to the QoS requirements. For example, the QoS requirements and measured elements include delay statistics of packets, throughput, and/or packet loss. At step 1020, the measured QoS elements and the QoS requirements are mapped to a satisfaction indicator (or measure) according to a satisfaction model. For example, a plurality of functions of the QoS elements and requirements (e.g., functions based on the difference between QoS elements and requirements) are first used to calculate a plurality of QoS satisfaction values. The values are then combined to calculate the satisfaction indicator. Alternatively, a single function of the QoS elements combined is used to calculate the satisfaction indicator. The satisfaction value can be a single value (a scalar) or a vector of values. The mapping can also be based on UBS/USC, by selecting or adjusting the mapping functions accordingly. At step 1030, user satisfaction is determined according to the satisfaction indicator.

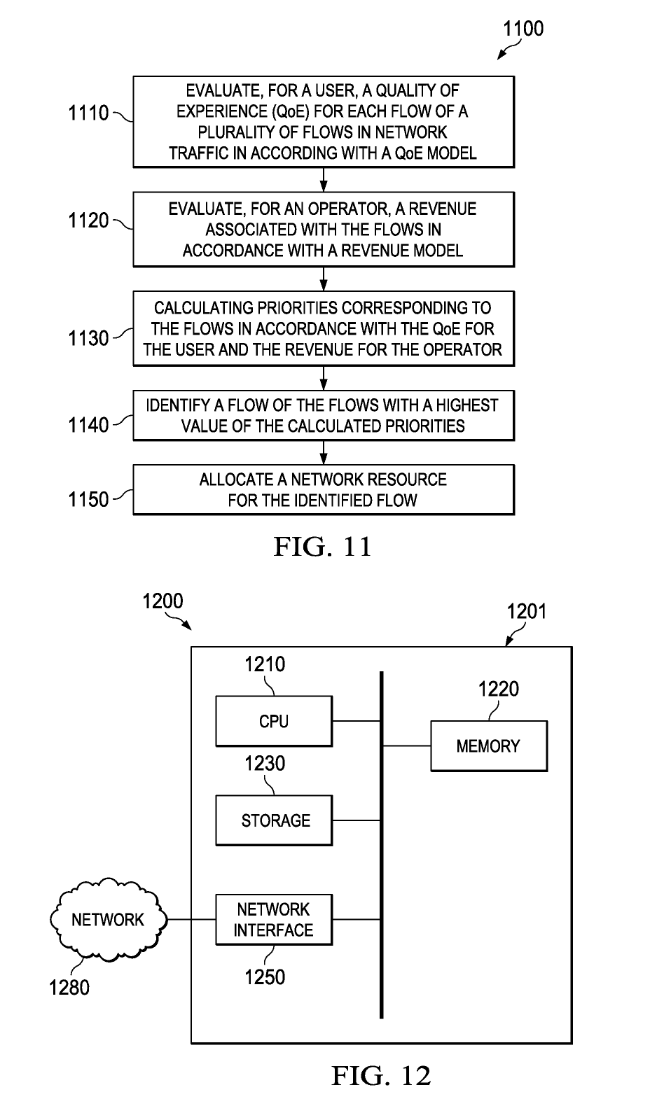

FIG. 11 illustrates an embodiment method 1100 for multi-objective joint packet/resource scheduling considering user satisfaction and fairness. At step 1110, a QoE is evaluated for a user for each flow of a plurality of flows in network traffic in according with a QoE model. At step 1120, a revenue is evaluated for an operator associated with the flows in accordance with a revenue model. At step 1130, priorities corresponding to the flows are calculated in accordance with the QoE for the user and the revenue for the operator. The priorities can be further based on channel conditions associated with the flows, current buffer status of the user, and a QoE fairness model defined by the operator. The QoE fairness model includes a metric to improve satisfaction fairness for QoE and quality of service (QoS) among multiple customers, and optionally a second metric to improve QoE and QoS fairness among the customers within a same user behavior or subscriber class. At step 1140, a flow with a highest value of the calculated priorities is identified. At step 1150, a network resource for the identified flow is allocated.

FIG. 12 is a block diagram of an exemplary processing system 1200 that can be used to implement various embodiments. For instance, the system 1200 may be part of a network component, such as a base station, a relay, a router, a gateway, or a controller/server unit. Specific devices may utilize all of the components shown, or only a subset of the components and levels of integration may vary from device to device. Furthermore, a device may contain multiple instances of a component, such as multiple processing units, processors, memories, transmitters, receivers, etc. The processing system 1200 may comprise a processing unit 1201 equipped with one or more input/output devices, such as a network interfaces, storage interfaces, and the like. The processing unit 1201 may include a central processing unit (CPU) 1210, a memory 1220, and a storage device 1230 connected to a bus. The bus may be one or more of any type of several bus architectures including a memory bus or memory controller, a peripheral bus or the like.

The CPU 1210 may comprise any type of electronic data processor. The memory 1220 may comprise any type of system memory such as static random access memory (SRAM), dynamic random access memory (DRAM), synchronous DRAM (SDRAM), read-only memory (ROM), a combination thereof, or the like. In an embodiment, the memory 1220 may include ROM for use at boot-up, and DRAM for program and data storage for use while executing programs. In embodiments, the memory 1220 is non-transitory. The storage device 1230 may comprise any type of storage device configured to store data, programs, and other information and to make the data, programs, and other information accessible via the bus. The storage device 1230 may comprise, for example, one or more of a solid state drive, hard disk drive, a magnetic disk drive, an optical disk drive, or the like.

The processing unit 1201 also includes one or more network interfaces 1250, which may comprise wired links, such as an Ethernet cable or the like, and/or wireless links to access nodes or one or more networks 1280. The network interface 1250 allows the processing unit 1201 to communicate with remote units via the networks 1280. For example, the network interface 1250 may provide wireless communication via one or more transmitters/transmit antennas and one or more receivers/receive antennas. In an embodiment, the processing unit 1201 is coupled to a local-area network or a wide-area network for data processing and communications with remote devices, such as other processing units, the Internet, remote storage facilities, or the like.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

1. A fusion protein comprising:
an immunogen sequence including an influenza A virus matrix protein M2e domain, or a fragment thereof, and an influenza A virus hemagglutinin fusion peptide (FP) domain, or a fragment thereof; and
an immunopotentiator sequence.

2. The fusion protein of claim 1, wherein the FP domain is from influenza A virus hemagglutinin subtype 1, 2, 3, 4, 5, 6, 7, 8, 9, 10, 11, 12, 13, 14, 15, 16, 17, or 18.

3. The fusion protein of claim 1, wherein the FP domain and the M2e domain are independently from an influenza A virus selected from an H1N1 virus, an H1N2 virus, an H2N2 virus, an H3N2 virus, an H5N1 virus, an H7N2 virus, an H7N3 virus, an H7N7 virus, an H7N9 virus, an H9N2 virus, or an H10N8 virus.

4. The fusion protein of claim 1, wherein the amino acid sequence of the FP domain is at least 90% identical to one of SEQ ID NOs. 3, 7, 17, 18, 19, or 20.

5. The fusion protein of claim 1, wherein the amino acid sequence of the M2e domain is at least 90% identical to one of SEQ ID NOs. 4, 8, 21, 22, 23, or 24.

6. The fusion protein of claim 1, wherein the protein further comprises a stabilization sequence.

7. The fusion protein of claim 4, wherein the stabilization sequence is a foldon (Fd) or GCN4 sequence.

8. The fusion protein of claim 1, wherein the immunopotentiator sequence is an Fc fragment sequence of human IgG Fc, a C3d sequence, an Onchocerca volvulus ASP-1 sequence, a cholera toxin sequence, or a muramyl peptide sequence.

9. The fusion protein of claim 1, wherein the immunopotentiatior sequence is at least 90% identical to the human IgG Fc sequence of SEQ ID NO. 10.

10. (canceled)

11. The fusion protein of claim 1, wherein the immunogen comprises M2e-FP or FP-M2e.

12. (canceled)

13. The fusion protein of claim 1, wherein the immunopotentiator sequence is linked to the C-terminus of the immunogen sequence.

14. The fusion protein of claim 1, wherein the stabilization sequence is linked to the C-terminus of the immunogen sequence, and the immunopotentiator sequence is linked to the C-terminus of the stabilization sequence.

15. The fusion protein of claim 11, wherein the fusion protein comprises M2e-FP-Fc, FP-M2e-Fc, M2e-FP-FdFc, or FP-M2e-FdFc.

16.-18. (canceled)

19. The fusion protein of claim 1, wherein the fusion protein further comprises a linker sequence disposed in at least one location from between the M2e and FP domains of the immunogen sequence, between the immunogen sequence and the stabilization sequence, and between the stabilization sequence and the immunopotentiator sequence.

20. The fusion protein of claim 19, wherein the linker is (GGGGS)ₙ, wherein n is an integer between 0 and 8.

21. (canceled)

22. An immunogenic composition comprising the fusion protein of claim 1 and at least one pharmaceutically acceptable excipient.

23. The immunogenic composition of claim 16 wherein the immunogenic composition further comprises an adjuvant.

24. A method of inducing an immune response against an influenza virus in a subject comprising:
administering the immunogenic composition of claim 16 to the subject.

25. The method of claim 19, wherein the immune response is a protective immune response.

26. The method of claim 19, wherein the influenza virus is an influenza A virus, an influenza B virus, or an influenza C virus.

27.-29. (canceled)
